(19) United States
(12) Patent Application Publication
(10) Pub. No.: US 2009/0143794 A1
(43) Pub. Date: Jun. 4, 2009

(54) Tissue resection device

(76) Inventors: Sean P. Conlon, Loveland, OH (US); Christopher Paul Swain, London (GB); Per-Ola Park, Goteborg (SE)

Correspondence Address: K&L GATES LLP, 535 SMITHFIELD STREET, PITTSBURGH, PA, 15222, US

(21) Appl. No.: 11/998,370

(22) Filed: Nov. 29, 2007

(51) Int. Cl. A61B 17/32; A61L 2/00

(52) U.S. Cl. 606/167; 422/28

(57) ABSTRACT

A surgical instrument which can be utilized to hold, view, transect, and suture tissue. The surgical instrument can include a tissue-receiving cavity which can be placed in fluid communication with a vacuum source such that the tissue can be held in the cavity. The surgical instrument can further include a housing and a cutting element, wherein the cutting element can be moved relative to the housing to resect the tissue positioned within the cavity. The surgical instrument can also include a passage configured to receive a suture applicator which can be moved between first and second positions to incise the tissue. In at least one embodiment, the passage can be configured to align and orient the suture applicator relative to the tissue positioned within the cavity. The surgical instrument can further include a passage configured to slidably receive an endoscope where the passage can align and orient the endoscope.

### BACKGROUND

1. Field of the Invention

The present invention generally relates to surgical instruments for resecting and/or incising tissue.

2. Description of the Related Art

In various circumstances, tissue may become diseased or may otherwise need to be removed from a patient's body. Previous surgical instruments have included a cutting member which can be utilized to transect such tissue. In various embodiments, these instruments have included one or more jaws configured to hold the tissue in place while it is cut. These jaws, however, require room to open and close. While useful for their intended purpose, these surgical instruments may not be suitable for use in certain surgical techniques, especially in techniques where there is little or no room for the jaws to move. In order to properly position such surgical instruments within a surgical site, an endoscope may also be positioned within the surgical site to observe the position of the surgical instrument. In such circumstances, a surgeon may have to independently manipulate the surgical instrument and the endoscope at the same time. While suitable in various circumstances, such a technique may require the surgeon to have significant skill and dexterity and/or require additional medical personnel to hold the various instruments. Furthermore, various surgical techniques may require the surgeon to suture, or otherwise fasten, the tissue after it has been resected. In such circumstances, the surgeon may have to position yet another surgical instrument, i.e., a suture applicator, for example, in the surgical site and hold this instrument relative to the other instruments. In other circumstances, the surgeon may have to remove a surgical instrument from the site and replace it with the suture applicator. In either event, these surgical techniques may be difficult or cumbersome to perform. What is needed is an improvement over the foregoing.

### SUMMARY

In at least one form of the invention, a surgical instrument can be utilized to hold, view, transect, and/or suture tissue. Such an instrument can reduce the amount of surgical instruments that are placed in a surgical site. In various embodiments, the surgical instrument can be placed in fluid communication with a vacuum source such that the surgical instrument can be used to grab and manipulate soft tissue, for example. In at least one embodiment, the surgical instrument can include a tissue-receiving cavity which can be placed in fluid communication with the vacuum source such that the tissue can be held in the tissue-receiving cavity. In at least one embodiment, the surgical instrument can further include a housing and a cutting element, wherein the cutting element can be moved relative to the housing and/or an end effector of the surgical instrument. In at least one such embodiment, the housing can include an actuator operably engaged with the cutting element wherein the actuator can be configured to move the cutting element between first and second positions to transect the tissue positioned within the tissue-receiving cavity. Such embodiments can be configured to allow the surgeon to hold the housing and operate the cutting element with one hand.

In various embodiments, the surgical instrument can further include a passage configured to slidably receive an endoscope wherein the surgical instrument can align and orient the endoscope relative to the targeted tissue. In various embodiments, the endoscope can be moved between a first position for viewing the surgical site and a second, retracted position. In at least one embodiment, the endoscope can be positioned within the tissue-receiving cavity when it is in its first position such that the endoscope can prevent, or at least inhibit, tissue from entering the cavity. In such embodiments, the endoscope can be retracted, or at least partially retracted, into its second position and allow the vacuum to draw the tissue into the tissue-receiving cavity. In various embodiments, the endoscope can be used to observe the tissue within the cavity while the endoscope is in its second position.

In various embodiments, the surgical instrument can further include a passage configured to receive another surgical instrument, such as a suture applicator, for example, which can be moved between first and second positions to incise the tissue. In at least one embodiment, the passage can be configured to align and orient the suture applicator relative to the tissue positioned within the tissue-receiving cavity. After the tissue has been incised, a suture anchor can be deployed from the suture applicator to secure a suture to the soft tissue. A second suture anchor and/or knotting element can then be deployed in order to fasten portions of the tissue together.

### BRIEF DESCRIPTION OF THE DRAWINGS

The above-mentioned and other features and advantages of the various embodiments of this invention, and the manner of attaining them, will become more apparent and the invention itself will be better understood by reference to the following description of embodiments of the invention taken in conjunction with the accompanying drawings, wherein:

FIG. 1 is a perspective view of a surgical instrument in accordance with an embodiment of the present invention;

FIG. 2 is a side view of the surgical instrument of FIG. 1;

FIG. 3 is a side view of an end effector of the surgical instrument of FIG. 1;

FIG. 4 is a top view of the surgical instrument of FIG. 1;

FIG. 5 is a bottom perspective view of the surgical instrument of FIG. 1;

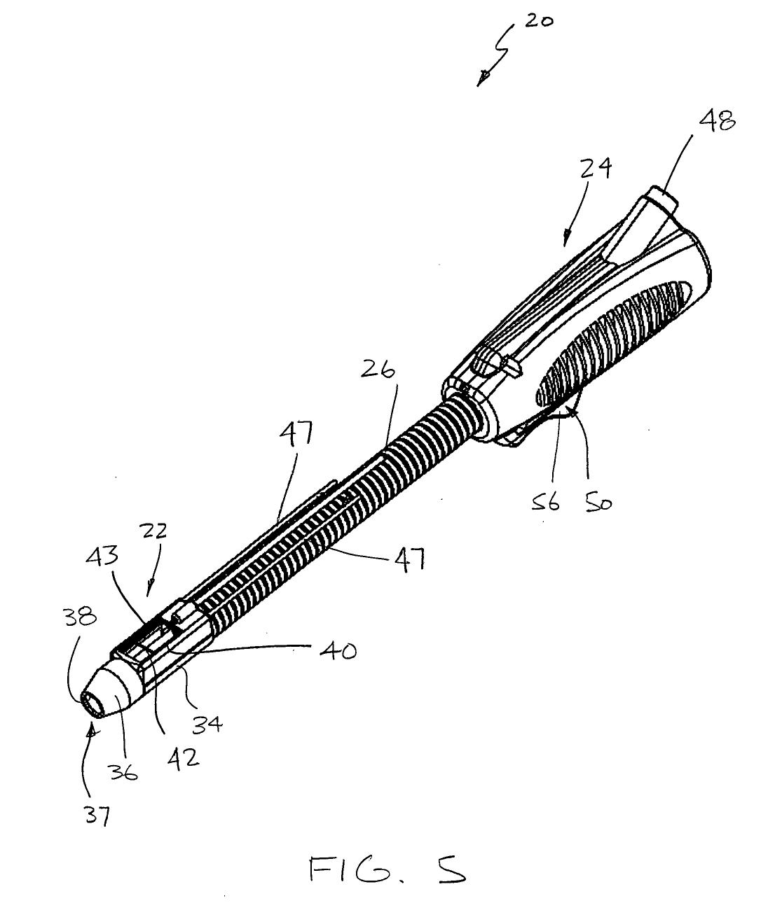

FIG. 6 is a perspective view of the end effector of the surgical instrument of FIG. 1;

FIG. 7 is a perspective view of the end effector of FIG. 6 with some portions of the surgical instrument removed;

FIG. 8 is an additional perspective view of the end effector of FIG. 6;

FIG. 9 is a side view of a hand piece of the surgical instrument of FIG. 1;

FIG. 10 is a perspective view of the hand piece of FIG. 9;

FIG. 11 is a perspective view of the hand piece of FIG. 9 with some portions of the surgical instrument removed;

FIG. 12 is a side view of the hand piece of FIG. 9 with some portions removed to illustrate a seal configured to receive a vacuum source connector;

FIG. 13 is a perspective view of the hand piece of FIG. 9 with additional portions of the surgical instrument removed;

FIG. 14 is a side view of the hand piece as illustrated in FIG. 13; and

FIG. 15 is a diagram illustrating soft tissue positioned within a tissue-receiving cavity in the end effector and a suture applicator positioned within a passage of the surgical instrument.

Corresponding reference characters indicate corresponding parts throughout the several views. The exemplifications set out herein illustrate preferred embodiments of the invention, in one form, and such exemplifications are not to be construed as limiting the scope of the invention in any manner.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

The various embodiments described herein are related to tissue resection devices. The devices described herein, and the surgical techniques for using the same, may be employed to treat tissue masses, tissue tumors, and lesions, for example, (all of which are hereinafter referred to as ‘diseased tissue’) at a tissue treatment region. In various embodiments, these devices can be utilized in open surgical procedures as well as external and non-invasive medical procedures. In other various embodiments, these devices and techniques may be adapted to provide minimally invasive access to the tissue treatment region or anatomic location, such as large and/or small intestine tissue, for example, in order to diagnose and treat the condition at the tissue treatment region more accurately and effectively. In various embodiments, portions of the devices may be introduced in the tissue treatment region endoscopically (e.g., laparoscopically and/or thoracoscopically) through a trocar extending through a small incision. Portions of other devices may be introduced into the tissue treatment region by way of a natural orifice through a cannula or catheter. Minimally invasive procedures which introduce medical instruments to a tissue treatment region through a natural opening of the patient are known as Natural Orifice Translumenal Endoscopic Surgery (NOTES)™. In other embodiments, portions of the devices can be introduced percutaneously or in any combination of the methods described above.

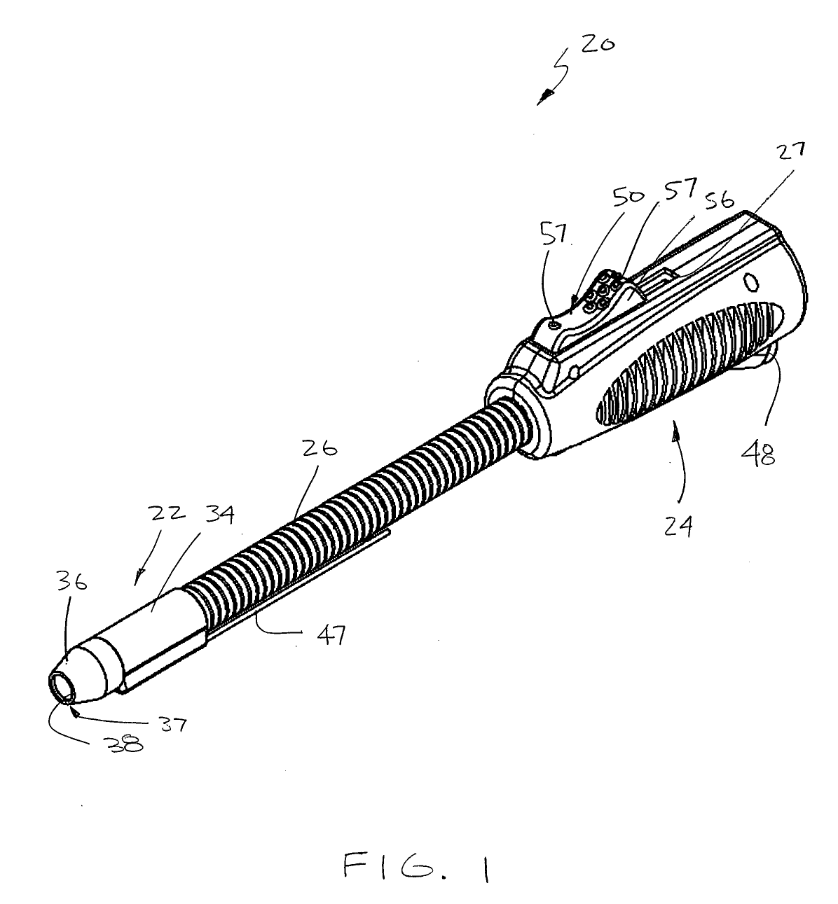

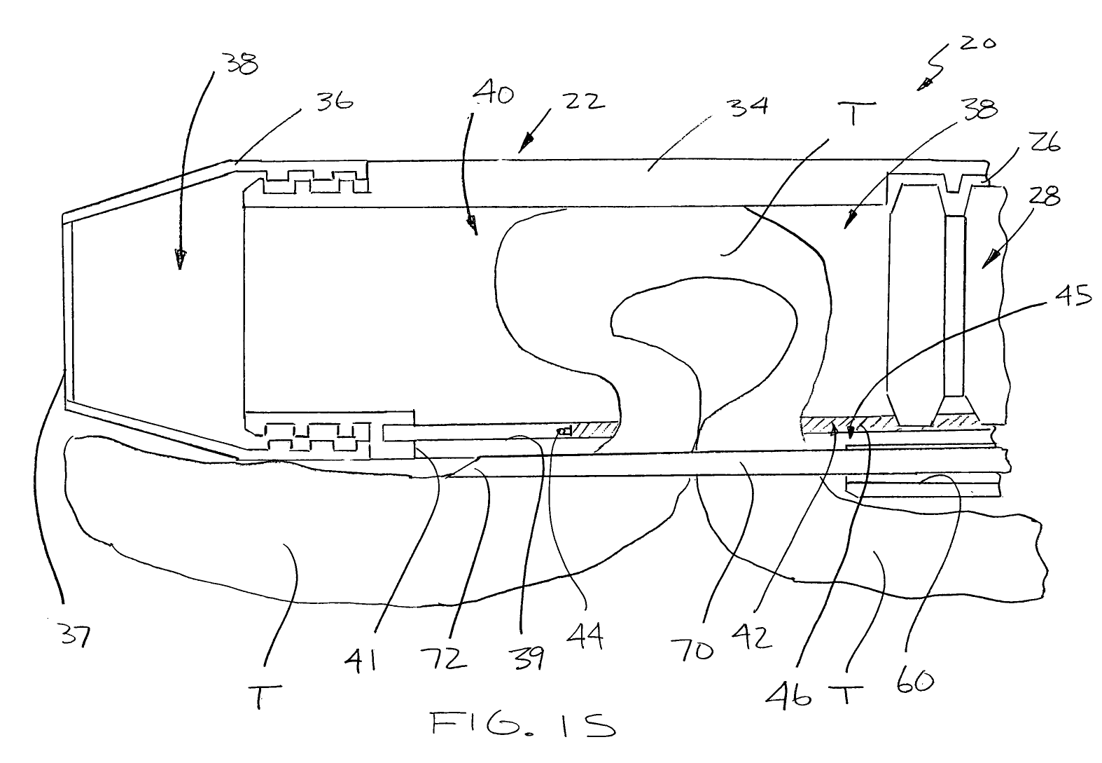

In various embodiments, referring to FIG. 1, a surgical instrument, such as surgical instrument 20, for example, can be inserted into a surgical site. In at least one embodiment, surgical instrument 20 can include end effector 22 and housing 24 wherein housing 24 can be configured to be grasped by a surgeon, for example, and end effector 22 can be configured to be positioned within the surgical site. In use, a trocar can be inserted into a patient and end effector 22 can be inserted through the trocar to access the surgical site. In at least one such embodiment, handle 24 can remain positioned outside of the surgical site and/or trocar such that handle 24 can be manipulated by a surgeon. Surgical instrument 20 can further include overtube 26 extending between end effector 22 and housing 24 where, referring to FIG. 15, overtube 26 can include aperture, or passage, 28 extending therethrough. In various embodiments, overtube 26 can be flexible and, in at least one embodiment, passage 28 can be configured to receive an endoscope therein. In at least one embodiment, the endoscope may include a flexible endoscope, such as a GIF-100 model available from Olympus Corporation, for example. In various embodiments, the endoscope may comprise a flexible shaft where the distal end of flexible shaft may comprise a light source, a viewing port, and at least one working channel. In at least one such embodiment, the viewing port can transmit an image within its field of view to an optical device such as a charge coupled device (CCD) camera within the endoscope, for example, so that an operator may view the image on a display monitor (not shown).

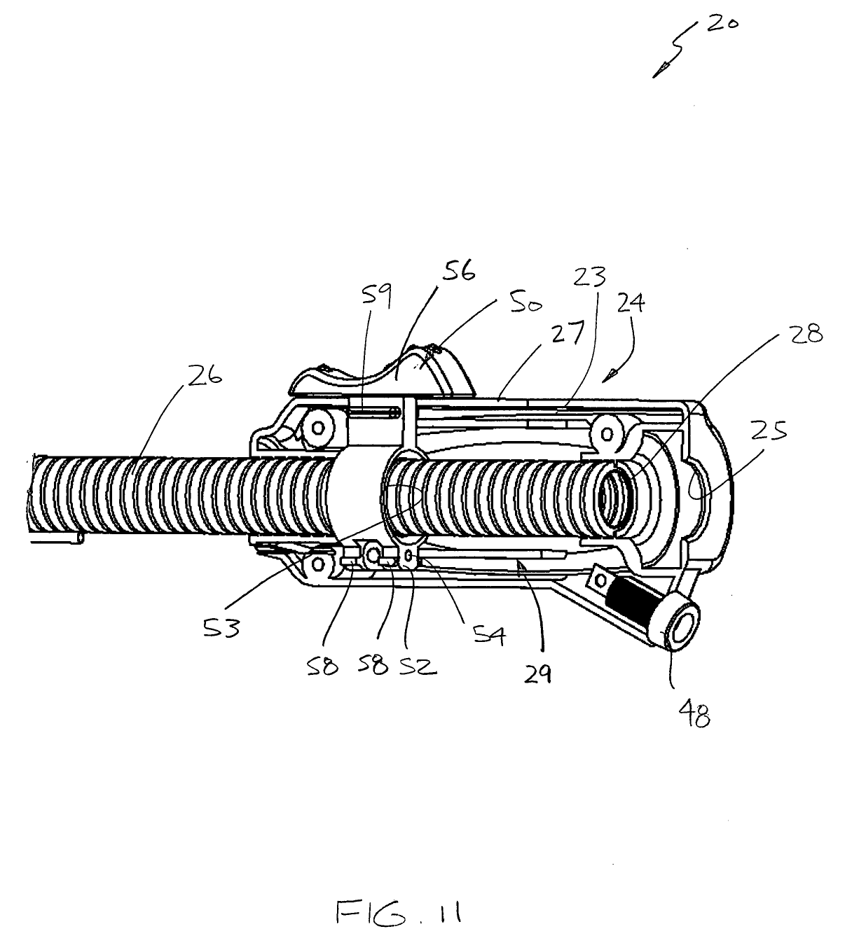

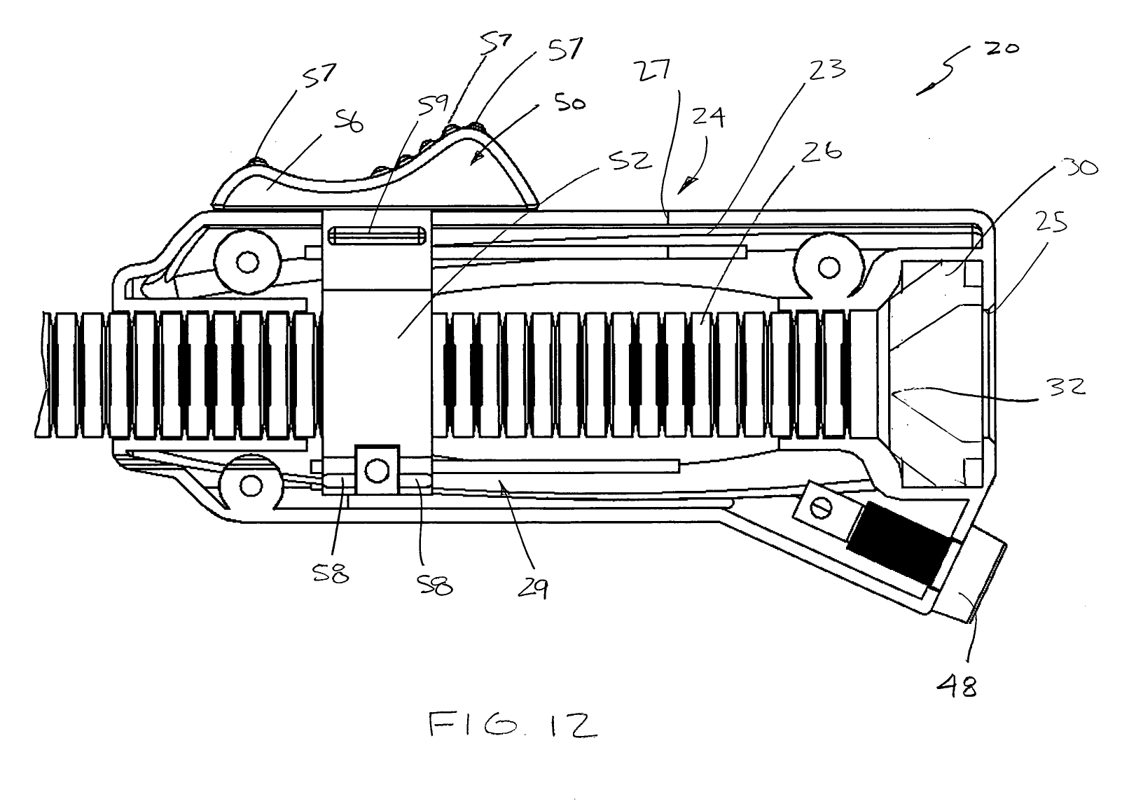

In at least one embodiment, referring to FIG. 11, the endoscope can be inserted into surgical instrument 20 through aperture 25 in housing 24. Thereafter, referring to FIG. 12, the endoscope can be positioned against seal 30 where, in at least one embodiment, a surgeon can apply a force to the endoscope to push the endoscope through aperture 32 in seal 30. In various embodiments, seal 30 can be configured to apply a compressive force against the endoscope such that seal 30 can sealingly engage the endoscope. After the endoscope has passed through seal 30, the endoscope can then be moved, or slid, into passage 28 (FIG. 15) of overtube 26. In at least one embodiment, passage 28 can be configured such that there is clearance between the endoscope and the sidewalls of passage 28. In other various embodiments, passage 28 can be configured such that there is a snug, or compression, fit therebetween. In either event, the endoscope can be slid within passage 28 until the distal end of the endoscope enters end effector 22.

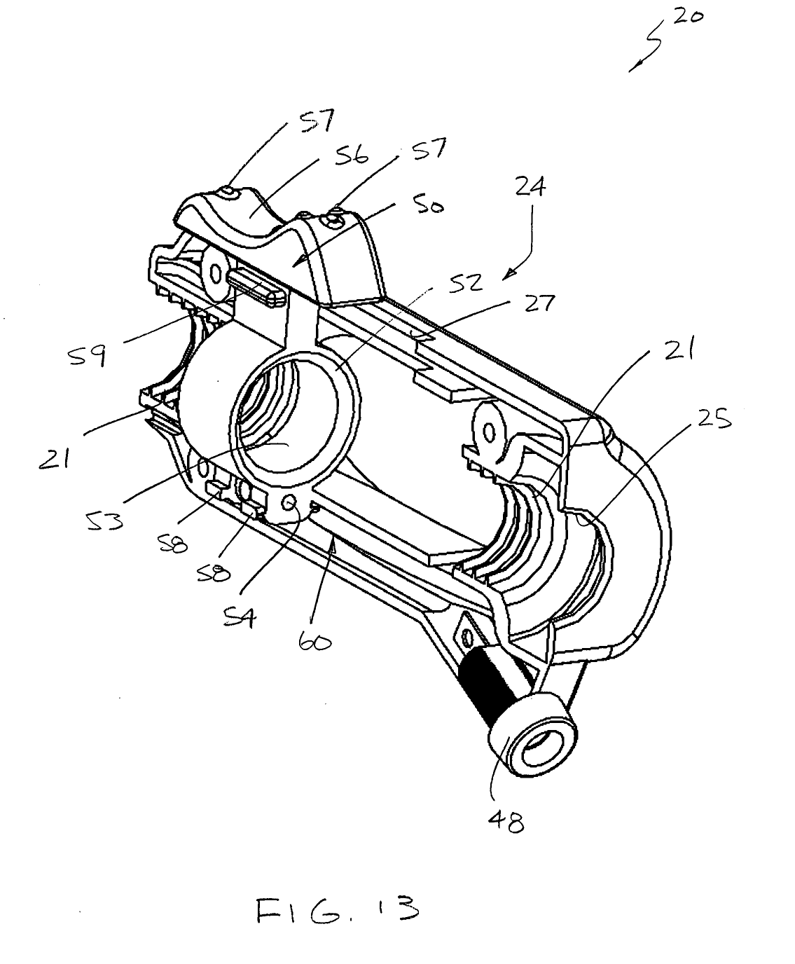

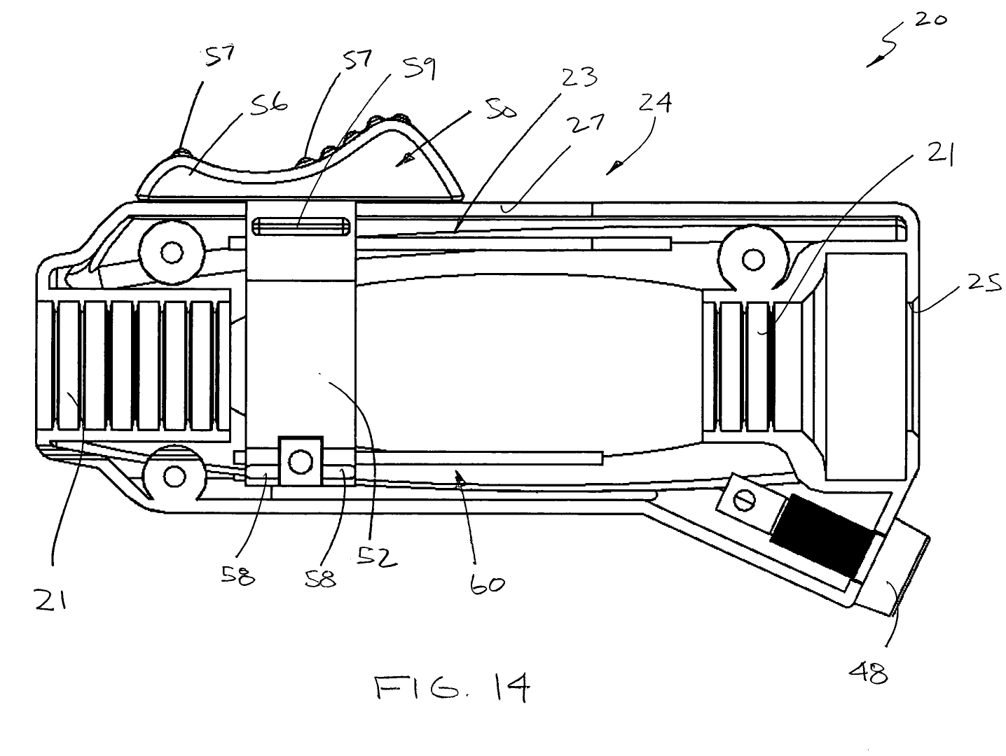

In various embodiments, referring primarily to FIGS. 1 and 15, end effector 22 can include housing 34 and nose cap 36, wherein cap 36 can be threadably connected to, or otherwise suitably connected to, housing 34. In other various embodiments, cap 36 can be integrally formed with housing 34. In either event, housing 34 and cap 36 can define aperture 38, where aperture 38 can also be configured to receive the endoscope. In at least one embodiment, the endoscope can be moved, or slid, distally within aperture 38 until the targeted tissue in the surgical site, for example, can be seen with the endoscope. In the event that the targeted tissue can't be seen with the endoscope, at least one of the surgical instrument and the endoscope can be manipulated within the surgical site. In at least one embodiment, as a result of the endoscope being positioned within surgical instrument 20, surgical instrument 20 can be manipulated to reposition and/or reorient the endoscope. In any event, in various embodiments, the endoscope can be rotated relative to end effector 22 and/or overtube 26, for example. In at least one such embodiment, the endoscope can be rotated such that the image displayed on the monitor, as described above, is conveniently oriented for the surgeon. In other various embodiments, the endoscope can be restrained from substantially rotating relative to the surgical instrument. In at least one such embodiment, the endoscope and at least one of end effector 22, housing 24, and overtube 26 can include cooperating key and groove features, for example, which can limit, or even prevent, relative rotational movement therebetween. In various embodiments, though, the cooperating key and groove features can permit relative translational movement therebetween. In any event, referring to FIGS. 13 and 14, housing 24 can include retaining portions 21 which can be configured to retain overtube 26 to housing 24. More particularly, in at least one embodiment, retaining portions 21 can include ridges and/or grooves which can be configured to cooperate with corresponding ridges and/or grooves on overtube 26 such that overtube 26 can be prevented, or at least inhibited, from being translated relative to housing 24. In at least one such embodiment, however, retaining portions 21 can permit relative rotational movement between overtube 26 and housing 24.

In various embodiments, cap 36 can include features which can permit an endoscope to extend beyond distal end 37 of cap 36 and at least partially exit aperture 38. In at least one embodiment, cap 36 can be at least partially comprised of a flexible, or resilient, material wherein aperture 38 can be configured to expand and allow the endoscope to pass therethrough. In certain circumstances, cap 36 can be configured to apply a compressive force to the endoscope positioned therein and create a seal therebetween. In various circumstances, cap 36 can include a seal which can seal, or at least substantially seal, aperture 38 when the endoscope is not positioned therein. In at least one such embodiment, cap 36 can be at least partially comprised of a silicone material, for example. In other various embodiments, cap 36, for example, can include features which can prevent the endoscope from being extended beyond distal end 37. In at least one such embodiment, cap 36 can include at least one inwardly extending ridge, or projection, which can act as a stop when the endoscope abuts the projection. In either event, after the surgeon has aligned the surgical instrument relative to the target tissue, the endoscope can be retracted proximally away from distal end 37. In effect, as described in greater detail below, the surgeon can move the endoscope between a first position and a second position within the surgical instrument.

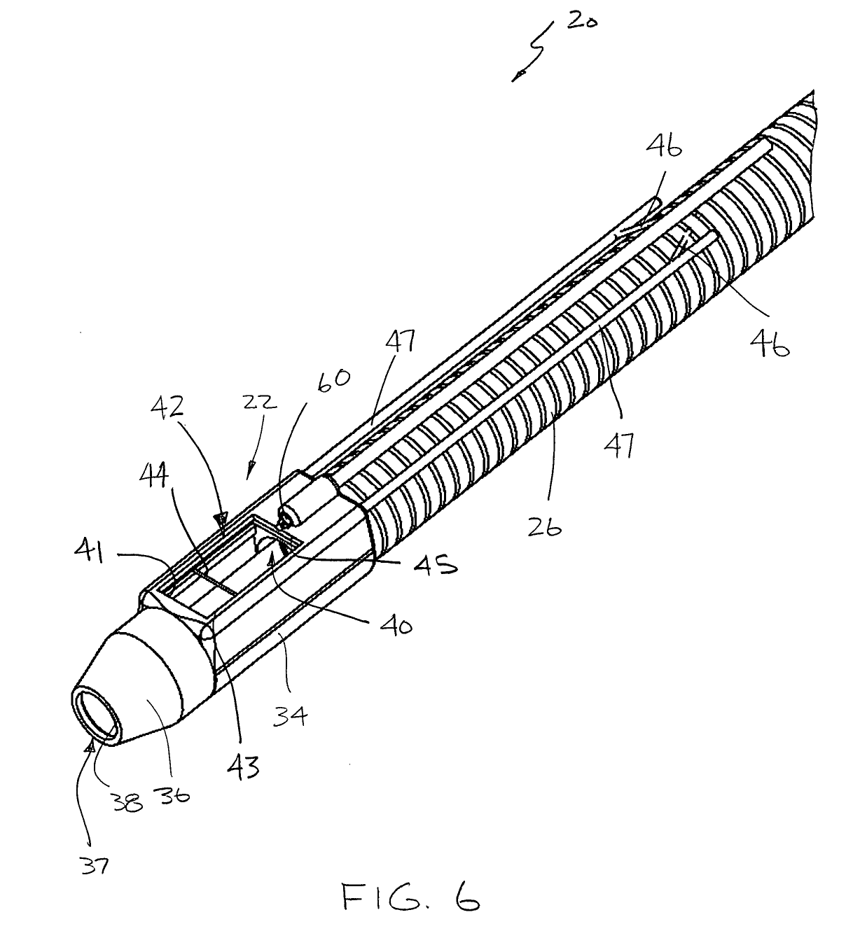

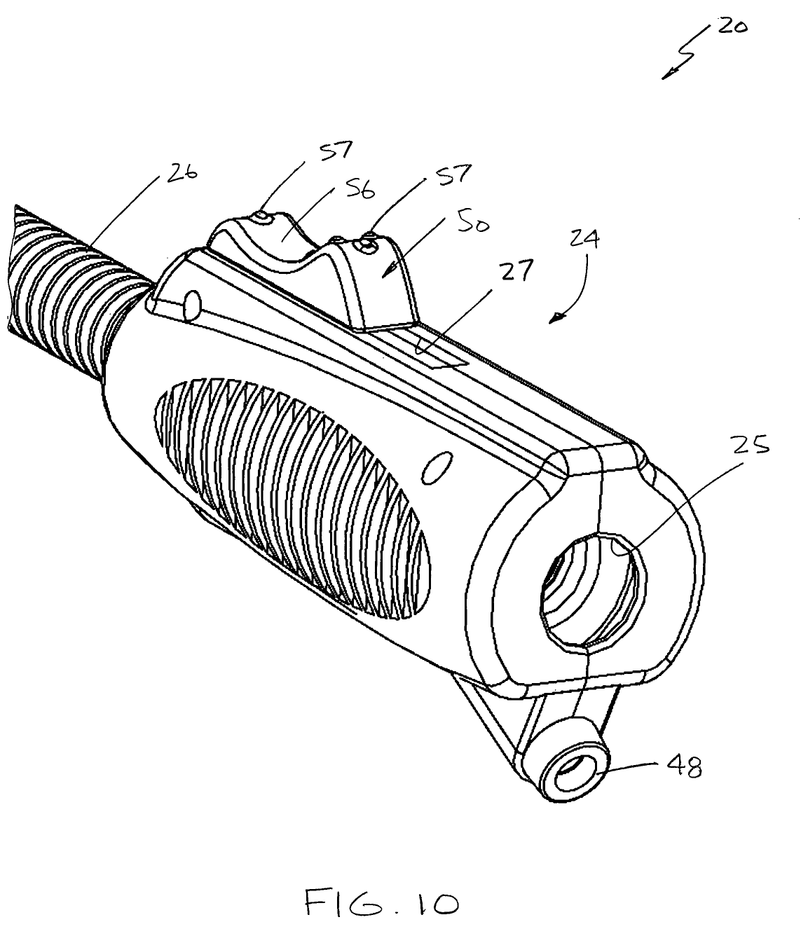

In various embodiments, referring to FIGS. 6 and 15, end effector 22 can further include tissue-receiving cavity 40. In at least one embodiment, tissue-receiving cavity 40 can be in fluid communication with aperture 38 in end effector 22 and, in addition, aperture 38 can be in fluid communication with passage 28 in overtube 26. In at least one such embodiment, a vacuum source can be operably connected with the endoscope such that the vacuum can be communicated into at least one of tissue-receiving cavity 40, aperture 38, and passage 28. In various alternative embodiments, although not illustrated, a vacuum source can be operably connected with housing 24 via aperture 25 (FIG. 10), for example, such that the vacuum can be communicated to tissue-receiving cavity 40. In either event, the vacuum source can include a switch which can be actuated by the surgeon, for example. Although not illustrated, surgical instrument 20 can include a switch which can be operated to allow the vacuum to be supplied to cavity 40. In various embodiments, the vacuum can, as a result of the above, draw the targeted tissue into cavity 40 and hold the tissue therein. In use, in at least one embodiment, the endoscope can substantially prevent tissue from entering into cavity 40 when the endoscope is positioned against and/or adjacent to distal end 37. In various embodiments, the endoscope can be retracted to allow the vacuum to pull the tissue into cavity 40. In at least one such embodiment, a seal can close, or at least substantially close, aperture 38 when the endoscope is withdrawn to further control the vacuum draw and assure that the targeted tissue is drawn into cavity 40. In either event, the endoscope can then be positioned to allow the surgeon to observe the tissue positioned within cavity 40. As described in greater detail below, once the targeted tissue, for example, is positioned within tissue-receiving cavity 40, the tissue can be resected or otherwise incised.

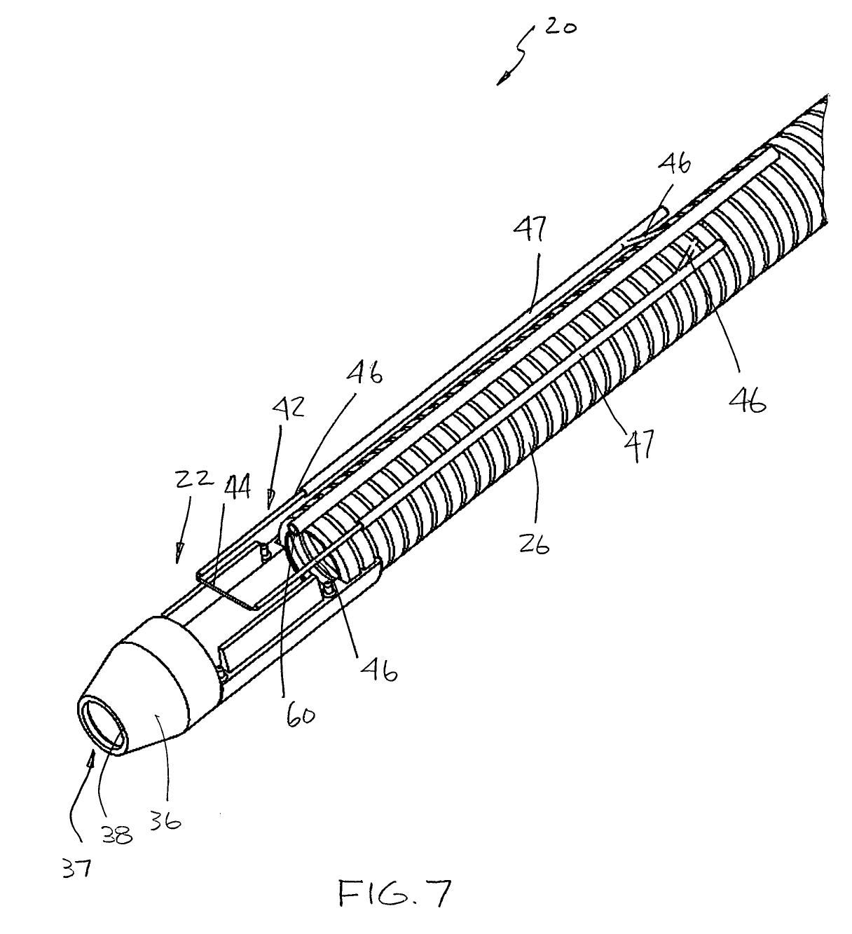

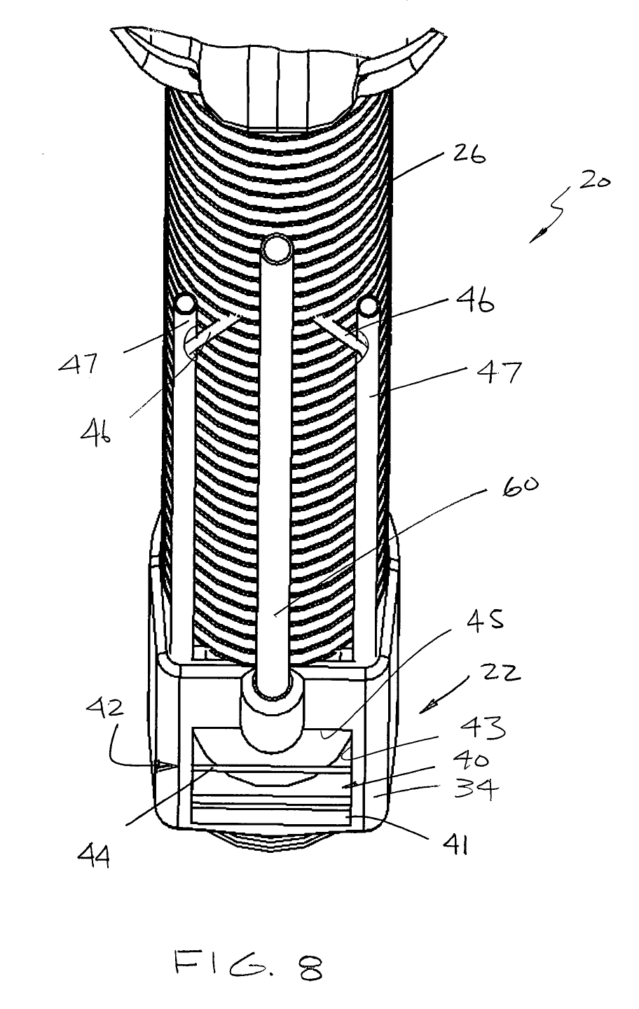

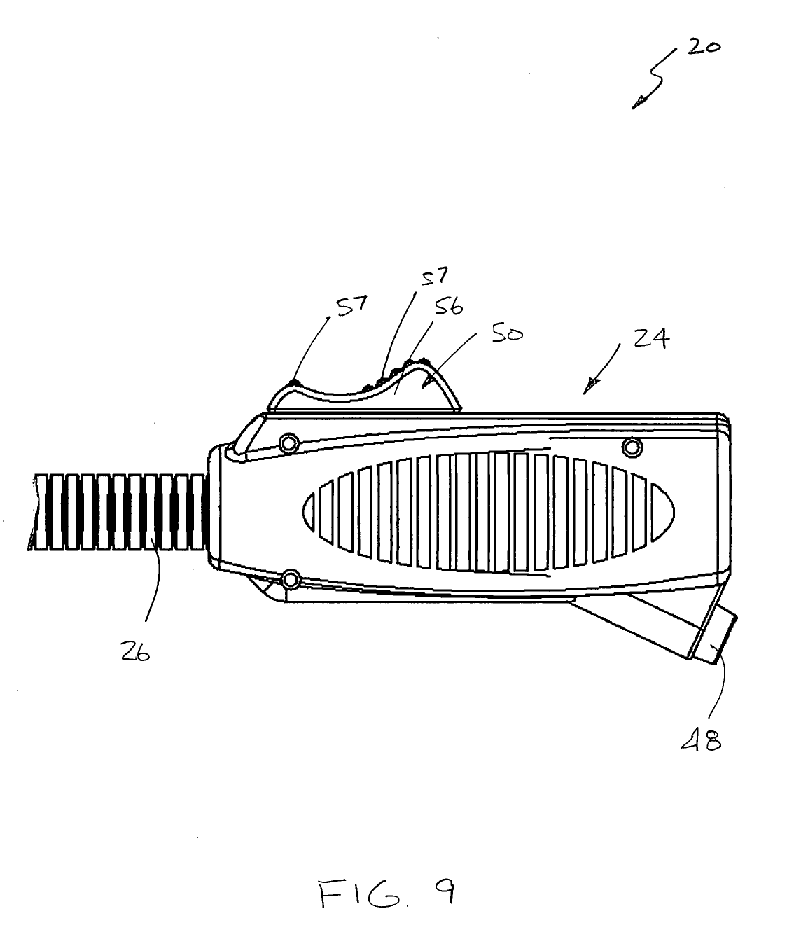

In various embodiments, a surgical instrument in accordance with at least one form of the present invention can include a cutting element operably engaged therewith. In at least one embodiment, referring to FIGS. 6, 7, 8, and 15, the cutting element, such as cutting element 42, for example, can be operably engaged with end effector 22 and/or housing 24 such that cutting element 42 can be moved relative to end effector 22 and/or housing 24. In at least one such embodiment, cutting element 42 can include a conductor, such as a metal wire, or snare, 44 which can be moved between first and second positions to incise the tissue “T” (FIG. 15) held within cavity 40. As described in greater detail below, snare 44 can be placed in electrical communication with an electrical source, such as an RF power source, for example, such that snare 44 can be energized and can cut the tissue as snare 44 is moved relative thereto. In at least one embodiment, referring to FIGS. 9-11, housing 24 can further include electrical connector 48 which can be placed in electrical communication with the electrical source and, in addition, cutting element 42 can further include conductors 46 (FIGS. 7 and 8) which can place connector 48 in electrical communication with snare 44. In various embodiments, conductors 46 can be comprised of insulated wires. For example, each conductor can include an inner core comprised, of copper, brass, and/or aluminum, for example, and an outer jacket, or sheath, which can cover the inner core, wherein the outer jacket can be comprised of PVC or any other suitable polymer, for example. In at least one embodiment, referring to FIG. 15, end effector 22 and/or overtube 26, for example, can include at least one recess or slot 39 configured to receive at least a portion of cutting element 42 and guide cutting element 42 relative to tissue-entrance window 43 (FIG. 6).

In use, snare 44 can be positioned against or adjacent to distal edge 41 of tissue-receiving cavity 40, for example, such that, when tissue enters cavity 40 as described above, the tissue can be positioned intermediate snare 44 and proximal edge 45 of cavity 40. Thereafter, snare 44 can be pulled proximally toward proximal edge 45 such that snare 44 can abut the tissue. When snare 44 is energized by an electrical source as described above, snare 44 can incise the tissue. In at least one embodiment, snare 44 may be sufficiently energized to also cauterize the tissue. In various alternative embodiments, snare 44 can be positioned against or adjacent to proximal edge 45 when the tissue enters cavity 40 such that snare 44 can be pushed distally against the soft tissue. In any event, in various embodiments, a current and/or voltage generator can be utilized to supply electrical power to cutting element 42. In various embodiments, the generator can include a switch which can be actuated by the surgeon, for example. Although not illustrated, surgical instrument 20 can include a switch, for example, which can be operated to allow electrical power to be supplied to cutting element 42. In various embodiments, the cutting element can include at least one knife, blade, or any other suitable cutting surface in addition to or in lieu of the electrical cutting element described above.

Once resected, at least a portion of the resected tissue, i.e., the remnant tissue, can be retained in tissue-receiving cavity 40. In various embodiments, an endoscope positioned within surgical instrument 20 can include a working channel configured to receive a grasper, for example, wherein the surgeon can insert the grasper into cavity 40 through the working channel and grasp the resected tissue. In at least one embodiment, the endoscope can be positioned within instrument 20 to assist the surgeon to observe the relative position of the grasper and the resected tissue within cavity 40. In various circumstances, the surgeon can then withdraw the grasper and the resected tissue from the surgical site through the working channel of the endoscope. In other circumstances, the resected tissue may be too large to be pulled through the working channel, for example, and the surgeon may withdraw the endoscope and grasper from surgical instrument 20 at the same time. In at least one embodiment, the vacuum supplied to cavity 40 can be sufficient to hold the resected tissue therein while surgical instrument 20 is removed from the surgical site through the trocar. As a result, when surgical instrument 20 is removed from the surgical site, the resected, remnant tissue can be removed from the surgical site as well.

In various embodiments, the cutting element can be moved relative to the end effector by an actuator. Referring to FIGS. 1 and 9-14, surgical instrument 20 can further include actuator 50 operably connected to cutting element 42. In at least one embodiment, actuator 50, referring primarily to FIG. 11, can further include attachment portion 52, wherein conductors 46 (FIG. 15) can be secured within aperture 54 of attachment portion 52. In various embodiments, actuator 50 can further include trigger, or thumbpad, 56 which can be displaced by the surgeon to displace conductors 46 and snare 44. In various embodiments, thumbpad 56 can be moved proximally and/or distally by the surgeon by placing their thumb, or any other suitable finger, on thumbpad 56 and sliding thumbpad 56 relative to housing 24. In at least one embodiment, conductors 46 can have sufficient slack therein, especially between actuator 50 and electrical connector 48, such that conductors 46 do not impede, or at least substantially impede, the movement of actuator 50.

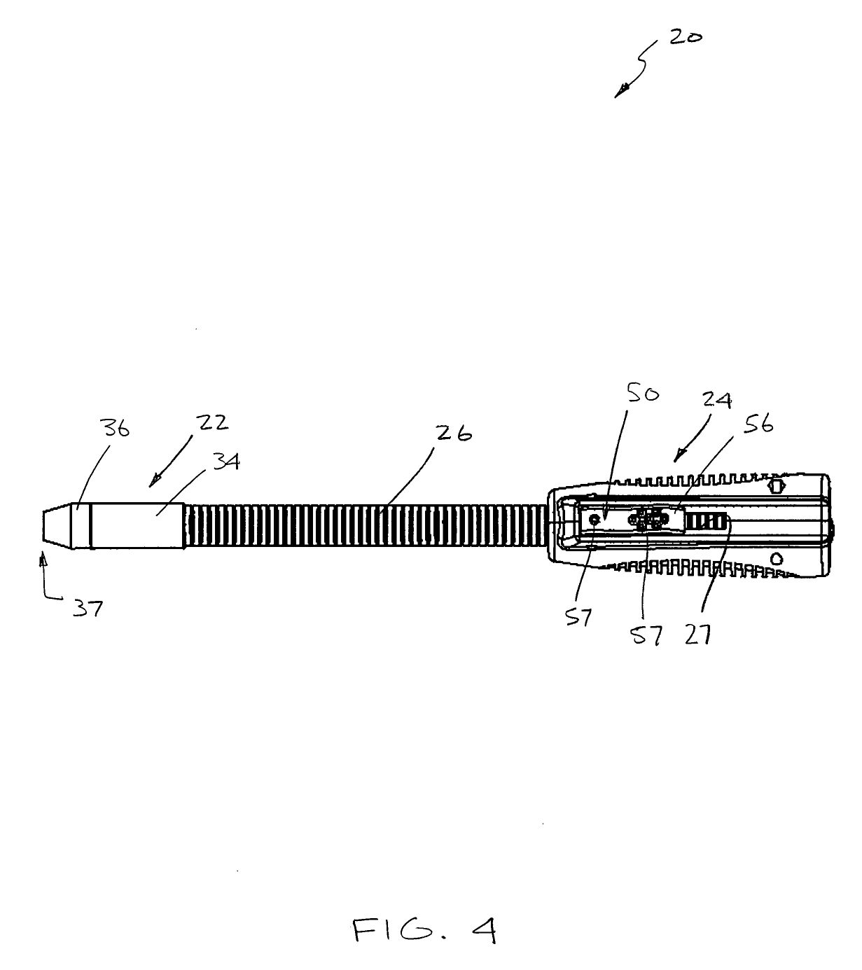

Further to the above, referring to FIGS. 1 and 4, housing 24 can further include slot 27 which can be configured to guide thumbpad 56 along a predetermined path. In at least one embodiment, slot 27 can define a substantially linear path such that thumbpad 56, and attachment portion 52 associated therewith, can be moved along a line between proximal and distal positions, for example. Although not illustrated, other suitable paths are contemplated including curved and/or curvilinear paths, for example. In various embodiments, referring to FIGS. 11-14, attachment portion 52 can include at least one lower projection 58 extending therefrom which can be configured to be slidably received in slot 29 in housing 24. In addition to or in lieu of projections 58, attachment portion 52 can further include at least one upper projection 59 extending therefrom, wherein projections 59 can be configured to be slidably received within slot 23 in housing 24. Although not illustrated, alternative embodiments are envisioned in which attachment portion 52, for example, includes a slot configured to receive at least one projection extending from housing 24. In various embodiments, referring to FIG. 1, thumbpad 56 can include at least one projection 57 extending therefrom which can be configured to prevent, or at least inhibit, a surgeon's thumb, for example, from slipping relative to the thumbpad 56. In at least one embodiment, thumbpad 56 can include any suitable contour and/or textured surface to facilitate the surgeon's use of actuator 50.

In various embodiments, referring to FIGS. 11-14, attachment portion 52 can include aperture 53, wherein aperture 53 can be configured to slidably receive overtube 26 such that attachment portion 52 can be moved relative to overtube 26. In at least one embodiment, referring to FIGS. 6-8, overtube 26 can further include at least one guide, such as guide 47, for example, which can be configured to slidably receive conductors 46. More particularly, in at least one embodiment, each guide 47 can be configured to receive a conductor 46 therein such that, when conductors 46 are slid relative to end effector 22 as described above, guides 47 can limit the movement of conductors 46. As a result of the above, in at least one embodiment, conductors 46 can remain positioned adjacent to the surgical instrument. In various embodiments, guides 47 can also be configured to at least partially insulate conductors 46 from soft tissue surrounding the surgical site, for example, and prevent, or at least inhibit, conductors 46 from unintentionally contacting the soft tissue.

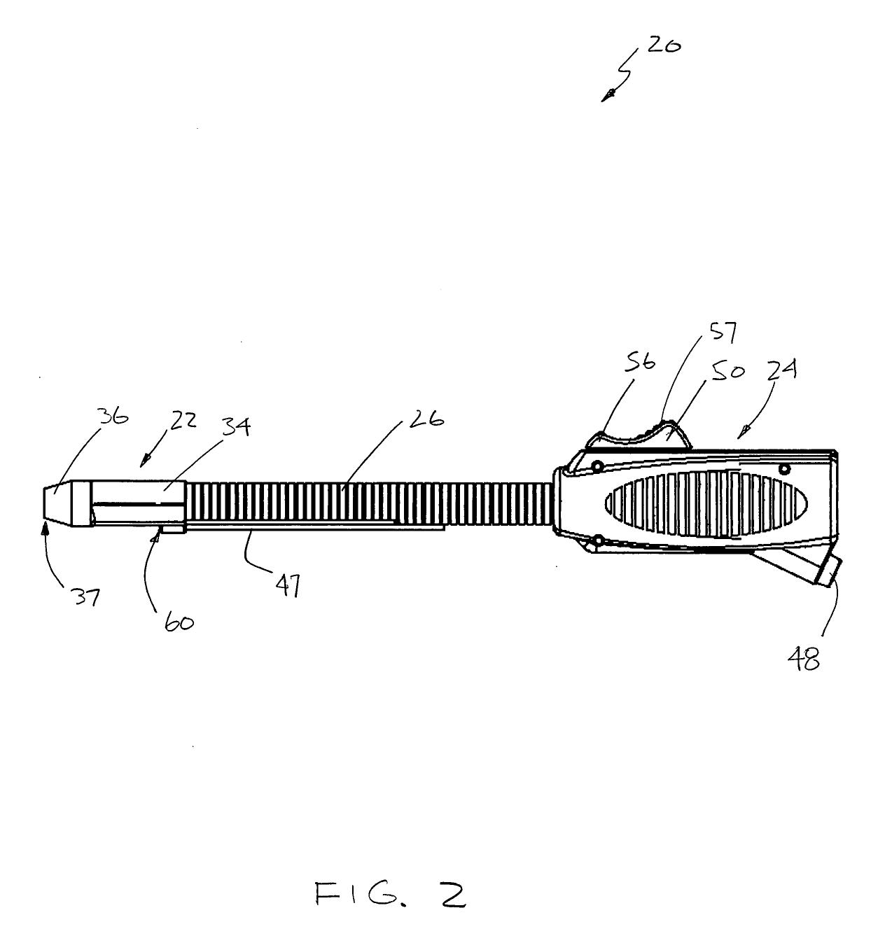

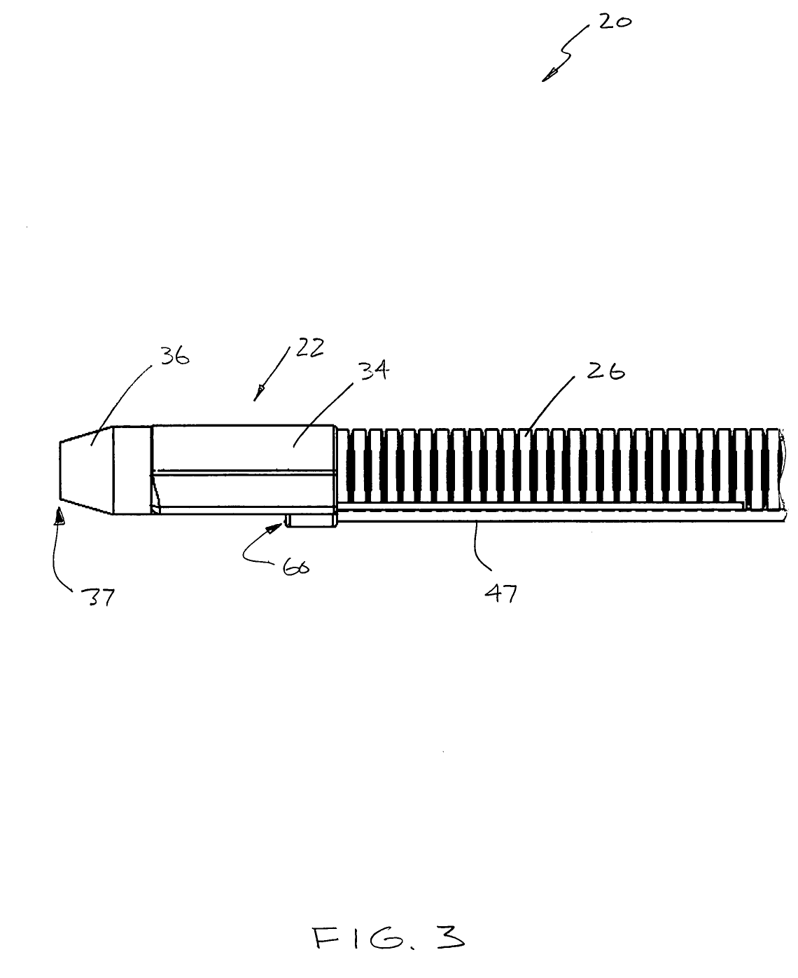

In various embodiments, a surgical instrument in accordance with at least one form of the present invention can include a working channel, or passage, configured to receive and/or guide a second, or additional, surgical instrument. In at least one embodiment, referring to FIGS. 2, 3, 6-8, and 15, surgical instrument 20 can further include passage 60 configured to slidably receive a surgical instrument, including a second cutting element, wherein passage 60 can be configured to align such a surgical instrument relative to surgical instrument 20. In at least one such embodiment, referring to FIG. 15, passage 60 can be configured to receive suture applicator 70 where suture applicator 70 can include incision end 72. In various embodiments, passage 60 can define an axis along which suture applicator 70, for example, can be guided. In at least one such embodiment, the axis can be oriented such that suture applicator 70 is guided toward tissue T. In use, once tissue T has been pulled into tissue-receiving cavity 40 as described above, suture applicator 70 can be moved relative to, or distally toward, tissue T such that incision end 72 can incise the tissue. In various embodiments, suture applicator 70 can be moved distally until end 72 exits the tissue. Thereafter, a suture anchor, such as a t-tag and/or knotting element, for example, can be deployed from suture applicator 70. In at least one embodiment, suture applicator 70 can include a cannula extending therethrough wherein the cannula can be configured to hold, or store, at least one suture and at least one suture anchor therein. In various embodiments, suture applicator 70 can further include a push rod which can be configured to be slid within the cannula and eject the suture anchor from the cannula.

After the suture anchor has been deployed, suture applicator 70 can be withdrawn proximally until end 72 exits the tissue, for example. A second suture anchor can then be ejected from the cannula and positioned relative to the tissue in order to capture the tissue between the first and second suture anchors. Other suitable suturing instruments and methods are disclosed in co-pending U.S. patent application Ser. No. 11/274,354, entitled SUTURE ANCHOR APPLICATOR, which was filed on Nov. 15, 2005, the entire disclosure of which is hereby incorporated by reference herein. Additional suitable suturing instruments and methods are disclosed in co-pending U.S. patent application Ser. No. 11/124,634, entitled DEVICES AND METHODS FOR ATTACHING AN ENDOLUMENAL GASTROINTESTINAL IMPLANT, which was filed on May 5, 2005, the entire disclosure of which is hereby incorporated by reference herein. In various embodiments, a suture can be inserted into the tissue before and/or after the tissue is transected by a cutting member as described above.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, various embodiments of the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

While this invention has been described as having exemplary designs, the present invention may be further modified within the spirit and scope of the disclosure. This application is therefore intended to cover any variations, uses, or adaptations of the invention using its general principles. Further, this application is intended to cover such departures from the present disclosure as come within known or customary practice in the art to which this invention pertains. Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A surgical instrument, comprising:
a housing including an aperture, wherein said aperture is configured to be placed in fluid communication with a vacuum source;
an end effector including a tissue-receiving cavity, wherein said tissue-receiving cavity is in fluid communication with said aperture such that the tissue can be held in said cavity by the vacuum source;
a cutting element movable relative to said end effector to transect the tissue; and
a passage configured to receive a suture applicator.

2. The surgical instrument of claim 1, wherein said aperture is configured to slidably receive an endoscope.

3. The surgical instrument of claim 2, wherein said aperture comprises a second passage extending between said tissue-receiving cavity and said housing, wherein said second passage is configured to slidably receive the endoscope therein.

4. The surgical instrument of claim 3, wherein said passage and said second passage are not in fluid communication with each other.

5. The surgical instrument of claim 1, wherein said tissue-receiving cavity is configured to retain at least a portion of the tissue therein after it has been transected.

6. The surgical instrument of claim 1, wherein said cutting element includes a conductor, wherein said conductor is configured to be placed in electrical communication with an electrical source, and wherein said conductor is configured to be moved relative to said end effector.

7. The surgical instrument of claim 6, wherein said conductor includes a metal wire.

8. The surgical instrument of claim 1, wherein said housing further includes an actuator configured to slide said cutting element between a first position and a second position.

9. The surgical instrument of claim 1, wherein at least one of said end effector and said housing includes said passage.

10. A method for processing an instrument for surgery, the method comprising:
obtaining the surgical instrument of claim 1;
sterilizing the surgical instrument; and
storing the surgical instrument in a sterile container.

11. A surgical instrument, comprising:
a housing;
a passage configured to slidably receive an endoscope, wherein said passage is configured to be placed in fluid communication with a vacuum source;
an end effector including a tissue-receiving cavity, wherein said tissue-receiving cavity is configured to selectively receive the endoscope, and wherein said tissue-receiving cavity is in fluid communication with said passage such that the tissue can be held in said cavity by the vacuum source;
a cutting element, wherein said cutting element is operably engaged with said housing and is movable relative to said housing to transect the tissue; and
a second passage configured to receive a suture applicator

12. The surgical instrument of claim 11, wherein said cutting element includes a conductor, wherein said conductor is configured to be placed in electrical communication with an electrical source, and wherein said conductor is configured to be moved relative to said end effector.

13. The surgical instrument of claim 11, wherein said tissue-receiving cavity is configured to retain at least a portion of the tissue therein after it has been transected.

14. The surgical instrument of claim 11, wherein at least one of said housing and said end effector further includes said second passage.

15. A method for processing an instrument for surgery, the method comprising:
obtaining the surgical instrument of claim 11;
sterilizing the surgical instrument; and
storing the surgical instrument in a sterile container.

16. A surgical instrument, comprising:
a housing including an aperture, wherein said aperture is configured to be placed in fluid communication with a vacuum source;
an end effector including a tissue-receiving cavity, wherein said tissue-receiving cavity is in fluid communication with said aperture such that the tissue can be held in said cavity by the vacuum source;
guide means for guiding a suture applicator; and
cutting means for cutting the tissue, wherein said cutting means is movable relative to said end effector.

17. The surgical instrument of claim 16, wherein said cutting means includes a conductor, wherein said conductor is configured to be placed in electrical communication with an electrical source, and wherein said conductor is configured to be moved relative to said end effector.

18. The surgical instrument of claim 16, further comprising a second passage extending between said aperture and said cavity, wherein said second passage is configured to slidably receive an endoscope therein.

19. The surgical instrument of claim 16, further comprising actuator means for actuating said cutting means.

20. A method for processing an instrument for surgery, the method comprising:
obtaining the surgical instrument of claim 16;
sterilizing the surgical instrument; and
storing the surgical instrument in a sterile container.
